(19) United States
(12) Patent Application Publication
(10) Pub. No.: US 2011/0144458 A1
(43) Pub. Date: Jun. 16, 2011

(54) Method and apparatus providing timed visual images during childbirth

(76) Inventor: Joseph Gauta, Naples, FL (US)

(21) Appl. No.: 12/839,850

(22) Filed: Jul. 20, 2010

Related U.S. Application Data
(60) Provisional application No. 61/286,154, filed on Dec. 14, 2009.

(51) Int. Cl. A61B 5/00

(52) U.S. Cl. 600/304

(57) ABSTRACT

Disclosed is an apparatus for providing a visual image timed to coincide with uterine contractions to assist a expectant mother during childbirth labor. The apparatus is particularly useful directed to help those expectant mothers using a regional anesthesia, such as an epidural anesthesia, in the second stage of labor to push effectively. The apparatus consists of a computer system that converts signals obtained from strategically positioned sensors such as a tocodynamometer or intrauterine pressure catheter into biofeedback visual vignettes that are understandable to the expectant mother. The signals are processed into the visual vignettes or a similar pictorial image based upon the pressure of the uterine contractions so that the expectant mother may understand her contraction pattern. This in turn will allow the expectant mother to visually understand when to start and stop pushing during the second stage of labor.

### PRIORITY CLAIM

This application is based upon and claims the priority filing date of U.S. Provisional Patent Application No. 61/286,154, filed Dec. 14, 2009, the contents of which are incorporated herein by reference.

### FIELD OF THE INVENTION

The invention generally relates to childbirth monitoring devices and in particular to an apparatus that transforms various physiological events such as the measured pressures of the uterus into a visual image, such as a vignette, for presentation to a expectant mother providing visual instruction when to push or stop pushing during the second stage of labor.

### BACKGROUND OF THE INVENTION

Childbirth is the process by which uterine contractions cause the fetus and placenta to be expelled from the uterus and birth canal. Rhythmic contractions of the uterine muscle create a force that pushes the fetus against the opening of the cervix. The cervix is a tubular structure that is firm and closed during pregnancy, keeping the baby and membranes protected inside the uterus. There are four stages of labor.

The first stage begins at the onset of labor and ends when the cervix is 100 percent effaced and completely dilated to 10 centimeters. Average length ranges for a first time mother up to 20 hours and shorter for subsequent births.

The second stage begins when the cervix is completely effaced and dilated and ends with the birth of the baby. Average length for a first time mother ranges from 1 to 2 hours and shorter for subsequent births.

The third stage begins with the birth of the baby and ends with the delivery of the placenta. Average length for the third stage of delivery ranges from 5 to 15 minutes. The fourth stage begins with delivery of the placenta and ends 6 weeks after delivery.

During the second stage, the cervix is completely effaced and dilated. By the use of contractions, the mother pushes the baby through the birth canal. Contractions are the forces that promote cervical dilation. Resistance of the cervix and the birth canal are the opposing forces to the contractions. In addition, the resistance of the cervix changes as it becomes more effaced and more dilated.

The sensation of extreme lower pelvic pain during the second stage of labor is the impetus to an important mechanism to shorten this stage, namely valsalva maneuvers, otherwise called pushing. Naturally, pushing helps to diminish the pain by altering some of the pain sensed by the patient.

The effacement, the dilation, the frequency of the contractions and the station are measured clinically during labor and are used by the doctors to determine if the labor is progressing normally. Hence, women in pain want to push, or need to push to decrease the pain of a uterine contraction. The desire to do this is innate. A regional anesthesia, such as an epidural anesthesia, spinal anesthesia, and saddle-block anesthesia could be used to obliterate the pain associated with the need to push. Pudendal nerve block is also an effective form of anesthesia that partially eliminates the pain associated with labor and hence part of the urge to push. Unfortunately, woman who undergo a regional anesthesia, such as an epidural, have such a decreased sensation of pain that they do not understand or sense the normal, evolutionary human response to the second stage of labor, namely when to push.

When the second stage of labor begins, the patient who is under the effect of an anesthesia has to rely on the medical professional, nurse, or doctor to tell her when to start pushing and when to stop. They also have to coach her on how to push and how hard to push. The anesthesia blunts the natural response to push, and therefore creates a situation in which the expectant mother does not respond to the natural urges to push. This creates a two-fold problem: 1) it requires external forces, namely coaching from support staff, to start and end the process successfully, and 2) it needlessly prolongs the second stage of labor, which can lead to detrimental effects to the mother and fetus.

With the advancement of technologies, a number of physiological conditions of the mother and baby during labor may also be monitored in order to determine the progress of labor. These additional conditions include: (1) effacement (the thinning out of the cervix that occurs before and during the first stage of labor); (2) cervical dilation (the increase in size of the cervical opening); (3) position of the cervix (the relation of the cervix to the vaginal axis, normally the fetal head); (4) station (the level of a predetermined point of the fetal presenting part with reference to the mother's pelvis); (5) position of the head which describes the relationship of the head to the pelvis; and (6) presentation which describes the part of the fetus (such as brow, face or breech) at the cervical opening. Such physiological conditions still rely upon an individual to measure and interpret against the monitors so that proper coaching is provided to the expectant mother.

A number of devices are currently employed for the purpose of monitoring a expectant mother, including U.S. Pat. No. 7,616,980 which discloses the use of electrode sensor array for maternal and fetal monitoring; U.S. Pat. No. 7,333,850 which discloses the use of maternal-fetal monitors and maternal uterine activity monitoring. U.S. Pat. No. 6,879,858 which discloses the use of electrodes for detecting uterine contractions; U.S. Pat. No. 5,483,970 discloses a electromyographic detection system. While these systems provide sensors, they fail to provide programming to convert the signals to a visual data display that are provided to the patient in a series of biofeedback vignettes that are understandable to the layman (a expectant mother with no medical background) and further illustrates the precise control of when to push or stop pushing.

U.S. Pat. No. 6,547,748 discloses a physiological event detector that improves upon the prior art by providing an audio indicator to inform a clinician as to the onset of a uterine contraction. This device is directed to the clinician and requires the clinician to instruct the patient to perform an action during the birthing event. U.S. Pat. No. 6,134,466 discloses a method of detecting true labor from detected electrical signals from muscular contraction and determining an average frequency of the signals. U.S. Pat. No. 5,301,680 discloses a diagnostic device using vector determination to determine uterine contractions and the extent of cervical dilation.

Devices also are used for providing biofeedback to patients with various medical problems. For instance, visual pictures to express the strength of anal and vaginal contractions are used in the field of urogynecology. Such devices are used in rehabilitative exercise to improve fecal and urinary incontinence in women.

The problem with the above is that the expectant mother must rely upon the professional staff, doctor, nurse coach or coaches, oral instructions which can lead to raised voices in the delivery room which adds to the stress of the event and possible miscommunications.

What is needed in the art is an apparatus that can rely on one or more physiological events and convert a signal into a visual image that is capable of invoking images to the expectant mother as to when to start pushing and when to stop pushing during labor.

### SUMMARY OF THE INVENTION

A computer system having a visual display is coupled to a biomedical process to help those patients with anesthesia in the second stage of labor to push effectively. Biofeedback is currently used in many physical rehabilitation settings to create better diagnostic and treatment modalities for illnesses. For example, it is currently being used in the setting of urinary and fecal incontinence to help improve embarrassing personal leaking and diminish the need for surgery. In a first embodiment, the instant invention may employ the use of a series of biofeedback loops, linked to a pressure transducer (tocodynamometer or intrauterine pressure catheter) of already existing fetal and/or contraction monitors in the hospital setting. The aim of this apparatus is to evaluate the pressure of the contractions and provide visual and auditory responses to the expectant mother so that she may understand her contraction pattern. This in turn will allow her to understand when to start and stop pushing during the second stage of labor, and will allow for improved medical and personal experiences. It will also help her to understand the series of events that allow her to push the hardest, thereby shortening this stage of labor. The invention can be incorporated directly into a tocodynamometer machine under an OEM scenario wherein the use of an additional monitor is employed to face the expectant mother and provide the visual responses so that she may understand the contraction pattern.

An objective of the instant invention is to empower the expectant mother by providing an apparatus that allows her to be in more control of the birthing process.

Another objective of the instant invention is to assist a expectant mother who has received an anesthesia to visually recognize the uterine contractions and effective timing information for pushing to support natural childbirth.

Another objective of the instant invention is to transform the measured pressures of the uterus to a visual vignette, or other image, for use in coaching the expectant mother during labor.

Another objective of the instant invention is to improve hospital efficiency by providing an apparatus that reduces staff time on the coaching aspect of childbirth and allow the staff to concentrate on other aspects of the labor process.

Yet another objective of the instant invention is to improve hospital efficiency by shortening the second stage thereby freeing up labor beds for future patients.

Still another objective of the instant invention is to improve patient satisfaction in her birthing experience and allow her to retain her strength by timing the need to push at the appropriate moment versus when there is no contraction or missing contractions.

Yet still another objective of the instant invention is to lower the confusion and stress level during a childbirth session typically found when a coach is used to inform the expectant mother when to push, as the emotions of the moment can lead to raised voice levels leading to confusion and stress for all involved.

Yet another objective of the instant invention is to employ biofeedback techniques coupled to a device capable of converting the biofeedback signals into visual vignettes and audio tones directed to the expectant mother.

Another objective of the instant invention is to modify an existing system, or incorporate during the manufacturing stage, that monitors the physiological condition of the expectant mother by modification of the system to produce visual vignettes in addition to the medical graphing illustration of the laboring effort.

Another objective of the instant invention is to teach the use of an audio portion that can be used in combination with the visual portion, turned on or off in accordance with the preference of the expectant mother, or be used independent of the visual portion using tones or music to encourage her and prompt her when to start and stop pushing.

Another objective of the instant invention is to provide a system that assists a expectant mother that does not use anesthesia to cope with and handle their pain better, and potentially shorten the second stage of labor.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objectives and features thereof.

### BRIEF DESCRIPTION OF THE DRAWINGS

FIG. 1 is a pictorial of a graphical display and expectant mother;

FIG. 2 is a block diagram of the apparatus system;

FIG. 3 is a pictorial of a vignette in an open position;

FIG. 4 is a pictorial of a vignette in a semi-exhausted position; and

FIG. 5 is a pictorial of a vignette in an exhausted position.

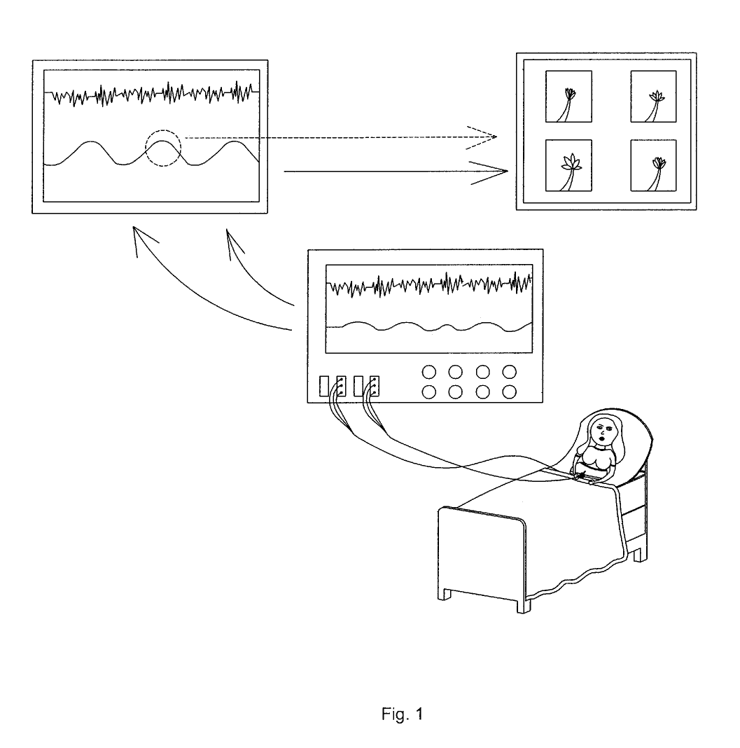

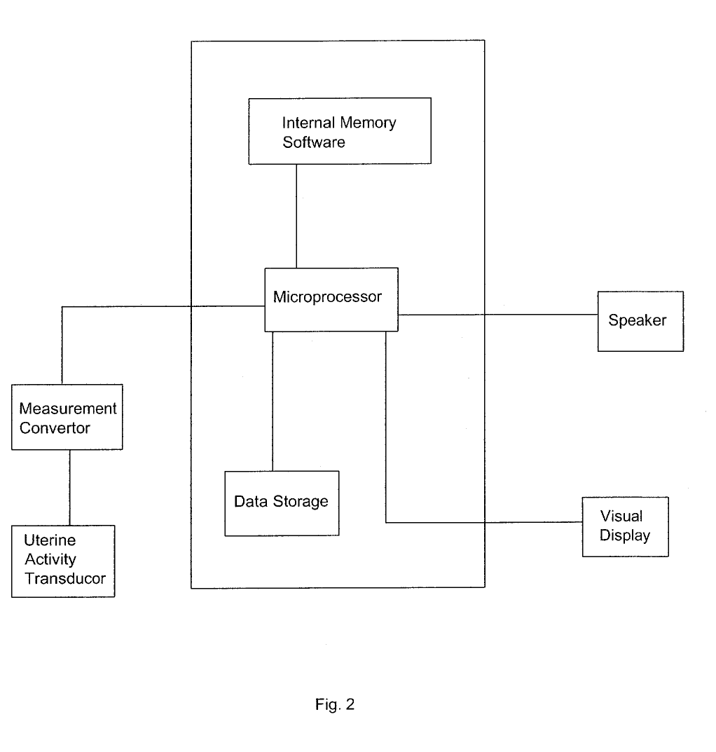

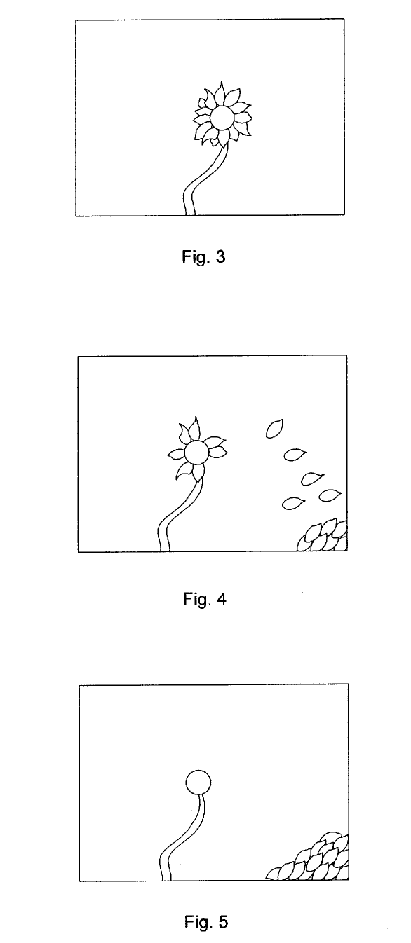

### DETAILED DESCRIPTION OF THE PREFERRED EMBODIMENT

The instant invention is based upon a computer system having a processor, a visual monitor, and a software program that is coupled to biomedical transducers. The software program may receive directional signals from existing childbirth equipment found at a birth center, namely equipment that is used to monitor the fetus and the maternal contraction pattern. The computer system consists of a monitor, either an LCD, plasma, or LED monitor will suffice being of a size that can be easily viewed by the expectant mother. If the monitor is placed proximate to the expectant mother, a 15″ monitor is sufficient. If the monitor is placed on a wall a distance from the expectant mother, a 60″ monitor might be more appropriate. It should be noted that a larger monitor allows others in the room to participate in the birthing process wherein contractions can be visually monitored and the expectant mother coached at the appropriate time. The monitor can be mounted on a pole, wall, or boom arm or in any position that provides a comfortable viewing position for the expectant mother to watch. The monitor is connected to the electronic fetal monitoring system via parallel/serial/USB cords or wireless transmission (e.g.—FM broadcast, Bluetooth technology, etc).

In the preferred embodiment, the existing childbirth equipment may include pressure transducers attached to the external surface of the mother on her belly (toco) or internally in the vagina and uterus (IUPC) which are used to measure the frequency, duration, and/or strength of each contraction. These pressure transducers convert these measurements into data that is usually displayed onto the box-like hardware that the nurses control and a monitor for larger viewing. Sometimes, the graphical data is even displayed outside of the birthing room onto larger monitors that many nurses can watch at the same time.

The expectant mother rarely gets to see or use this graphical representation of her contraction pattern unless she is trained in how to interpret the graphical representation of her contraction pattern, as the representation could lead to additional confusion and stress during the childbirth process.

The graphical data, usually conveyed visually as a series of bell curves on a monitor, is used by the medical personnel for timing of contractions. The patient has traditionally relied upon what the medical personnel have told her regarding when and how to push during her contractions. The patient has not traditionally been able to know or understand the pattern currently displayed to the clinician.

The monitor used in the instant invention is placed in front of the expectant mother so that she may be able to view specialized graphical data to help her push with contractions during the second stage of labor. Though intended to be used by expectant mothers with anesthesia, this product could be used by any expectant mother, regardless of anesthesia status. The graphical presentations currently provided are directed to trained medical personnel and systems fail to provide programming to convert the signals to a visual display that are provided to the patient in a series of vignettes, understandable to the layman (a expectant mother with no medical background).

A software program converts a measurement of the contractions which are preferably measured in mm Hg (millimeters of mercury, the unit of measurement). Measurements are proportional to the amount of pressure the uterine contraction creates or the amount of pressure the mother creates with her pushing forces (valsalva). Externally, the transducer senses the pressure produced by the uterine contraction, the mother's valsalva and abdominal muscle contraction, and skin. Maternal uterine activity is noted and recorded when the pressure of a contraction pushes on a sensor, which is on the underside of a tocodynamometer. The sensor on the tocodynamometer must be placed on that part of the uterus that can be palpated easily.

The internal pressure transducers are more accurate because they are placed into the uterus/womb and measure only the internal pressure of the uterus contraction, not the superfluous pressure of the mother's abdominal wall or skin. An internal monitor may be used to determine the actual strength of the contraction as well as the resting tone of the uterus. For instance, a woman may appear to be having strong contractions but not be progressing in labor. Thus, progress in labor is best determined by cervical dilation. The insertion of an intrauterine pressure catheter permits the determination of the strength of the contractions in millimeters of Hg, a measurement used for pressure. A good labor pattern that facilitates cervical dilation can be calculated by taking the difference in pressure between the peak of the contraction and the resting tone and adding them up over a ten-minute period. The unit of measurement for this calculation, is called a Montevideo unit, wherein ideally the sum total of the pressures should be between 150 and 250 Montevideo units to achieve cervical dilation. If the calculation is in this range and the woman's cervix is not changing, then and only then can a diagnosis of failure to progress be made. The intrauterine pressure catheter also provides an accurate measurement of the resting tone of the uterus. It is important that the uterus relax between contractions in order for the baby to receive oxygen. If the uterus is not relaxing or if the resting tone is rising it is an indication of possible placental abruption, the tearing away of the placenta from the wall of the uterus. Either way, whatever pressure is sensed by the transducer (internally or externally) is transmitted via wires to the electronic fetal monitoring system. This pressure measurement is now graphed onto paper and on a screen in the form of a bell curve/Gaussian curve.

Another use of an intrauterine pressure catheter is for amnioinfusion. This is a procedure in which a physiologic solution (such as normal saline) is infused into the uterine cavity to replace the amniotic fluid. It is used to relieve cord compression, reduce fetal distress caused by meconium staining, and as a correction of decreased amniotic fluid.

The peak of the uterine contractions coincides with the peak of the curve in mmHg, and the beginning and end of the uterine contractions coincide with the beginning and end of the curve. This resting tone is usually around 10-25 mmHg (baseline). Peak uterine contractions pressure is usually around 80 mmHg, occasionally as high as 100-110 mmHg (peak).

For example: employing a flower as a visual point, at a resting tone of 10-25 mmHg, a resting/baseline tone is realized by the sensor and a doctor or qualified nurse can determine the optimum baseline point. Whenever the uterus contraction pressure is at this level of 10-25 mmHg, an image of a flower is displayed on the screen, in this example a closed flower. Every 2-5 minutes, a new uterus contraction will start and stop, each one lasting about 1 minute long. During this 1 minute long uterus contraction, the pressure against the transducers changes. The pressure increases as the uterus contraction strengthens (this is when the expectant mother starts to feel uterus contraction pain), then it decreases as the uterus contraction weakens. The pressure of the uterus contraction may peak at around 80-100 mmHg. At this peak uterus contraction pressure, the flower is completely open. All the pressures during the uterus contraction are displayed on the monitor as changes in the “openness” of the flower. In other words, a petal of the flower could open with each sequential 10 mmHg increase in the pressure. Let's say that the flower is closed at a baseline pressure of 25 mmHg. At 35 mmHg, one of the petals opens. When a uterus contraction pressure of 45 mmHg is reached, another petal opens, and so on for each 10 mmHg gradient in pressure. At 80-100 mmHg uterus contraction pressure, the operator can signify the peak uterus contraction pressure. This alerts the system that most of the uterine contractions have peaked at around this pressure, and the system can adjust the pressure parameters for each petal. When the expectant mother is pushing, pressures may be higher. The goal of pushing with the instant invention monitoring the contractions may be to have the expectant mother valsalva and measure the pressures. When the expectant mother pushes beyond what the system is set to consider the peak uterus contraction pressure, the flower will start to disintegrate.

If the average peak pressure was 80 mmHg and at this time the flower petals were completely open. As the expectant mother produces valsalva and reaches 90 mmHg of pressure, the flower starts to bend and the sound of wind can be provided in an audio format. At 100 mmHg of valsalva, one of the flower petals may fly off the flower and the sound of wind is heard more loudly. At 110 mmHg, more petals fly off the flower and the wind starts to howl, at 120 mmHg, all the flower petals may fly off and just the stem is noted flapping in the howling wind.

These images are displayed on the monitor in a continuous, flowing pattern. The images are a continuum/spectrum of changes from closed to open to closed again. As the valsalva maneuvers and uterine contractions pressure decreases, the flower and the wind start to diminish. During the resting phase in between uterine contractions (usually about 2-5 minutes), the flower image is regenerated and ready for the next round of uterine contractions and valsalva.

While the flower scenario is an illustrative example, the type of image is left to one's imagination. For instance, a mother pushing a baby carriage up a hill or a child blowing out her 1^{st} birthday candles and many, many other images can be used to show the mother what her uterine contractions are doing.

In another example, when a contraction starts, a picture of a empty balloon could be presented on the screen. As the pressure on the internal or external transducer increases, the balloon could be opened proportionally by a child blowing air into the balloon. When the contraction fades and/or the mother's pushing efforts wane, the balloon starts to deflate. To keep the mother engaged, the vignettes could constantly change or the mother could pick the series of vignettes, e.g.—flowers opening, a rocket reaching the moon, the sun rising and setting, and so forth.

The actual hardware, the monitor, could be connected to the existing hardware via cords or Bluetooth. The monitor is positioned in a comfortable position for viewing by the laboring patient. While the medical personnel may continue to interpret data from the transducers, the instant software will convert the data into a cartoon-like effect as above or the like that can be easily interpreted by non-medical personnel. The more pressure that is sensed by the transducer (meaning that the mother is pushing harder), the more the vignette expands to an end point like a completely open flower.

The auditory and visual clues to the laboring patient are a key component of this invention. Biofeedback vignettes are used to allow the patient to know about her pushing efforts. This pressure gradient is what would be used to create changes in the visual and auditory clues to the patient. As the pressure changes from baseline, the vignette characters also would change. An example would be a line getting longer as the measured uterine pressure increases, and the line getting shorter as the uterine pressure decreases. This same idea would be used across all the vignettes.

The auditory component is also used to increase the biofeedback stimulation to the expectant mother. In the flower example, if she reaches pressures above 70 cmH2O, the sound of a howling wind would increase until her maximally reached pressure, at which time the howling wind subsides and the flower starts to close up again or starts to limp when another flower is reborn.

It should be noted that the use of a visual vignette has been described above as an exemplary embodiment; however, any type of visual presentation can be used both in animation as well as actual photos/films. This may include personalized photos/films of the expectant mother's immediate family or anything of interest stated by the expectant mother that might be used to lower the anxiety of the child birthing process. These items can be drawn from a preexisting catalog of items, or be created from personal effects to further enhance the laboring process. For instance, a personalized visual effect could be of the expectant mother blowing the candles on a birthday cake. If rate of pushing is proper the flame will bend appropriately; if the rate of pushing is insufficient the candle may not move; if the rate of pushing is excessive then the candles and cake may all begin to slide off a table. The use of the visual presentation is limited only by ones imagination. In addition, the technology employed in this invention can be wired or made wireless (e.g. Bluetooth) so as to limit the equipment necessary while enhancing the birthing process.

The system can be incorporated into existing systems currently using pressure transducers for monitoring a expectant mother, or as a stand-alone unit consisting of its own pressure transducers.

In another embodiment, family photos or videos of friends or family doing fun things can be incorporated into pleasing, flowing images visualized on the system at the time of labor. A web-based, proprietary software program would disassemble and reassemble the images to proportionally correspond to the pressure gradient of the uterine contractions. Personal images are more likely to make an impact on the expectant mother, and would more likely keep the attention of the expectant mother focused on the uterine contractions and what she is supposed to do.

A webcam can be attached to allow the expectant mother to communicate with her family that is otherwise not allowed in the birthing room or cannot be at the delivery with her. Skype or some other form of web-based communication services could be used to partner with the invention for added patient appeal. During the boring waiting period of the 1^{st} stage of labor (the beginning of uterine contractions causing cervical change to the time of complete cervical dilation at 10 cm), the expectant mother and partner can pick images, communicate with loved ones, pick voices and sounds to be displayed on the system, and so forth.

A Wi-Fi enabled system is yet another embodiment. Most hospitals have or can easily install a Wi-Fi connection in the facilities. Using passwords and secure connection internally, the expectant mother could communicate with her family members that are in the waiting room via iPhone apps, Windows based apps, smart phones, or any other type of instant text/messaging devices wherein the expectant mother can inform her family and friends of her condition, dilation, phase of labor, and so forth.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A medical apparatus for assisting a expectant mother during childbirth comprising:
a computer having a display monitor;
at least one sensor positioned on a expectant mother to acquire a uterine activity signal from a uterine contraction, said uterine activity signal coupled to said computer;
a software program for operating the computer to determine a change in the uterine activity signal and converting said signal into a vignette image viewable on said display monitor;
wherein said vignette image provides timing of the contraction pattern and visual instruction of when to start and stop pushing during labor by altering said vignette image.

2. The medical apparatus according to claim 1 wherein said uterine contraction are measurements proportional to the amount of pressure that the expectant mother's uterine contraction creates.

3. The medical apparatus according to claim 1 wherein said uterine contraction are measurements proportional to the amount of pressure the expectant mother creates through valsalva.

4. The medical apparatus according to claim 1 wherein said uterine contraction are measurements proportional to the amount of pressure the expectant mother creates through abdominal muscle contraction.

5. The medical apparatus according to claim 1 wherein said sensor is a tocodynamometer placed on the external surface of uterus in an area that can be palpated easily.

6. The medical apparatus according to claim 1 wherein said sensor is an internal pressure transducer placed into the uterus for measuring the internal pressure of the uterine contractions.

7. The medical apparatus according to claim 2 wherein a baseline measurement is used to visually depict a vignette in a first position that continues to change until a peak measurement is obtained placing said vignette in a second position.

8. The medical apparatus according to claim 7 wherein said vignette begins to disintegrate after said second position is obtained.

9. The medical apparatus according to claim 1 wherein an audio sound is produced when a preset peak measurement is reached, said audio sound increasing in tone as said measurement exceeds said peak.

10. The medical apparatus according to claim 7 wherein said vignette is displayed in a dynamic format in response to measurement change proportional to the pressure received by a transducer.

11. A medical apparatus for assisting a expectant mother during childbirth comprising:
a computer having a display monitor;
at least one internal pressure transducer placed into the uterus for measuring the internal pressure of the uterine contractions on a expectant mother to acquire a uterine activity signal from uterine contraction measurements that are proportional to the amount of pressure that the expectant mother's uterine contraction creates, said uterine activity signal coupled to said computer;
a software program for operating the computer to determine a change in the uterine activity signal and converting said signal into a vignette image viewable on said display monitor;
wherein said vignette image provides timing of the contraction pattern and visual instruction of when to start and stop pushing during labor by a altering said vignette image whereby a base measurement is used to visually depict a vignette in a first position that continues to change until a peak measurement is obtained placing said vignette in a second position.

12. The medical apparatus according to claim 11 including a sensor wherein said uterine contraction are measurements proportional to the amount of pressure the expectant mother creates through valsalva.

13. The medical apparatus according to claim 11 including a sensor wherein said uterine contraction are measurements proportional to the amount of pressure the expectant mother creates through abdominal muscle contraction.

14. The medical apparatus according to claim 11 wherein said tocodynamometer is placed on an area that can be palpated easily.

15. The medical apparatus according to claim 11 including a tocodynamometer sensor positioned placed onto the uterus for measuring the pressure of the uterine contractions.

16. The medical apparatus according to claim 11 wherein a preset peak measurement visually depicts said vignette disintegrating.

17. The medical apparatus according to claim 11 wherein an audio sound is produced when a preset peak measurement is reached, said audio sound increasing in tone as said measurement exceeds said peak.

18. The medical apparatus according to claim 7 wherein said vignette is displayed in a dynamic format in response to measurement change proportional to the pressure received by a transducer.
